SEQUENCE LISTING (1 sequence)

SEQ ID NO: 1 - DNA, 1527 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggtgaggccacaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

(19) United States
(12) Patent Application Publication
(10) Pub. No.: US 2022/0154139 A1
(43) Pub. Date: May 19, 2022

(54) YAP1 Gene-Modified Mesenchymal Stem Cell and Preparation Method Thereof

(71) Applicant: Zhejiang University, Hangzhou (CN)

(72) Inventors: Hongcui Cao, Hangzhou, Zhejiang (CN); Lanjuan Li, Hangzhou, Zhejiang (CN); Xudong Feng, Hangzhou, Zhejiang (CN); Jiong Yu, Hangzhou, Zhejiang (CN)

(21) Appl. No.: 17/588,289

(22) Filed: Jan. 30, 2022

Related U.S. Application Data
(63) Continuation-in-part of application No. PCT/CN2020/094898, filed on Jun. 8, 2020.

(30) Foreign Application Priority Data
Jan. 5, 2020 (CN) 202010007670.1

(51) Int. Cl. C12N 5/073; C12N 5/0775; C12N 15/86

(52) U.S. Cl. CPC C12N 5/0605; C12N 5/0667; C12N 2740/15043; C12N 15/86; C12N 2509/00; C12N 5/0668

(57) ABSTRACT

The present invention relates to the field of biotechnology and aims to provide a YAP1 gene-modified mesenchymal stem cell and a method for preparing the same. The mesenchymal stem cell is a primary mesenchymal stem cell modified by overexpressed YAP1 gene, wherein the YAP1 gene is derived from a YAP1 lentiviral vector or a YAP1 plasmid vector, and the primary mesenchymal stem cell is derived from any of the following human tissues: placenta, umbilical cord or adipose tissue. The YAP1 gene-modified mesenchymal stem cell obtained by the present invention has no effect on the phenotype and differentiation ability of MSCs themselves; the present invention can significantly promote the proliferation of mesenchymal stem cell and further increase the cell yield by modifying the mesenchymal stem cell with overexpressed YAP1 gene; therefore, a large number of mesenchymal stem cells can be rapidly obtained for clinical stem cell transplantation therapy.

### CROSS-REFERENCE TO RELATED APPLICATION

The present application is a Continuation-in-part Application of PCT Application No. PCT/CN2020/094898 filed on Jun. 8, 2020, which claims the benefit of Chinese Patent Application No. 202010007670.1 filed on Jan. 5, 2020. The contents of the above are hereby incorporated by reference in their entirety.

### REFERENCE TO SEQUENCE LISTING

A sequence listing is submitted as an ASCII formatted text filed via EFS-Web, with a file name of “Sequence_listing.TXT”, a creation date of Jan. 28, 2022, and a size of 2,426 bytes. The sequence Listing filed via EFS-Web is part of the specification and is incorporated in its entirety by reference herein.

### TECHNICAL FIELD

The present invention belongs to the field of biotechnology, and specifically relates to a YAP1 gene-modified mesenchymal stem cell and a method for preparing the same.

### BACKGROUD

Mesenchymal stem cells (MSCs) have become the most promising substitutes for embryonic stem cells in regenerative medicine and clinical therapy due to their ability of self-renewal and multilineage differentiation. In recent years, preclinical and clinical studies have successively demonstrated the significant therapeutic effects of MSCs in immune diseases, myocardial injury, liver disease, lung injury, kidney disease and diabetes mellitus, etc. MSCs may be isolated from a variety of tissues, such as bone marrow, placenta, adipose tissue, synovial tissue, lung tissue, umbilical cord blood and peripheral blood, etc. However, previous studies have shown that the optimal number of cells for a single implantation to treat liver disease is about 1-5×10⁷ regardless of the source of MSCs. Considering that clinical mesenchymal stem cell transplantation treatment requires a large number of cells and the initial number of mesenchymal stem cells obtained from tissues is small, primary MSCs need to be extensively expanded in vitro to meet the magnitude of infusion for clinical therapy. Therefore, the focus of research in regenerative medicine has always been on how to quickly obtain the number of cells that can be used for clinical cell transplantation.

The proliferative capacity of MSCs is influenced by many factors, including the source of individual and tissue, the culture conditions, and ongoing passages. Mesenchymal stem cells without genetic modification have a slower proliferation rate in vitro, and it may take a long time to expand to the magnitude of clinical application. Therefore, finding and cloning the relevant genes that regulate the proliferation capacity of mesenchymal stem cells will help to promote the industrialization of MSCs, but also help them to be widely used in clinical therapy.

### SUMMARY

The technical problem to be solved by the present invention is to overcome the deficiencies in the prior art and provide a YAP1 gene-modified mesenchymal stem cell and its preparation method, thereby meeting the requirements of clinical cell transplantation for a large number of cells.

The technical solution of the present invention is provided to solve the technical problem.

Provided is a YAP1 gene modified mesenchymal stem cell, which is a primary mesenchymal stem cell modified by overexpressed YAP1 gene.

In the present invention, the YAP1 gene is derived from a YAP1 lentiviral vector or a YAP1 plasmid vector. The YAP1 gene has a coding sequence of SEQ ID NO: 1.

In the present invention, the primary mesenchymal stem cell is derived from any of the following human tissues: placenta, umbilical cord or adipose tissue.

The present invention further provides a method for preparing the aforementioned YAP1 gene-modified mesenchymal stem cell, comprising:

(1) Taking small pieces of placenta, umbilical cord or adipose tissue containing a primary mesenchymal stem cell; washing with phosphate buffer saline (PBS) on a culture dish until a washing solution becomes clear, and then sufficiently cutting up;

(2) Transferring the chopped tissue pieces to a 50 ml centrifuge tube, adding 25 ml of collagenase IV at a mass/volume concentration of 0.1%, and digesting by shaking on a shaker at a constant temperature of 37° C. for 30 minutes;

(3) Adding 20 ml of phosphate buffer saline to the digested tissue, mixing well and filtering through a 100 pm sieve to obtain a filtrate; centrifuging the filtrate at 1200 rpm for 5 minutes to obtain a supernatant, and removing the supernatant;

(4) Adding 5 ml of DMEM medium containing 20% fetal bovine serum to the cell precipitate, mixing well and seeding into a T25 culture flask; then placing in an incubator with 5% CO₂ and a constant temperature of 37° C.; replacing with a fresh DMEM medium containing 20% fetal bovine serum every 3 days;

(5) After the cell have reached 50% confluence, adding a YAP1 lentiviral vector or a YAP1 plasmid vector at a multiplicity of infection of 50:1 to transfect the mesenchymal stem cell; after completing transfection, a primary mesenchymal stem cell YAP1-LV-MSC modified by overexpressed YAP1 gene is successfully prepared, wherein the YAP1 lentiviral vector or a YAP1 plasmid vector contains a YAP1 gene coding sequence of SEQ ID NO: 1.

A use of the gene-modified mesenchymal stem cell in cell expansion is characterized by increasing an expression of the protein encoded by the YAP1 gene through gene modification and improving a proliferation rate of mesenchymal stem cell.

The principle of the present invention is described as below:

The protein encoded by the YAP1 gene, known as YAP1 or YAP65, is a protein that serves as a transcriptional regulator by activating the transcription of genes involved in cell proliferation and repressing apoptotic genes, and has been reported to be used in cancer cell research. However, the use of the YAP1 gene for modifying mesenchymal stem cells has not been reported.

The YAP1 gene in the present invention is derived from a YAP1 lentiviral vector or a YAP1 plasmid vector (i.e., a lentiviral vector or plasmid vector containing a coding sequence of YAP1 gene).

Since the YAP1 gene allows mesenchymal stem cells to proliferate at a significantly faster rate, sufficient numbers of cells can be obtained for clinical cell transplantation in a short period of time. Therefore, the yield of mesenchymal stem cells may be rapidly increased.

The beneficial effects of the present invention compared with the prior art are provided as below.

1. The YAP1 gene-modified mesenchymal stem cell obtained in the present invention has no effect on the phenotype and differentiation ability of the mesenchymal stem cell themselves.

2. The present invention can significantly promote the proliferation of mesenchymal stem cells and further increase the cell yield, by modifying the mesenchymal stem cells with overexpressed YAP1 gene; therefore, a large number of mesenchymal stem cells can be obtained rapidly for clinical stem cell transplantation therapy.

### BRIEF DESCRIPTION OF THE DRAWINGS

FIG. 1 shows the relative expression level of YAP1 protein after overexpression.

FIG. 2 shows the growth curves of mesenchymal stem cells after YAP1 overexpression.

FIG. 3 shows the population doubling time of mesenchymal stem cells after YAP1 overexpression.

FIG. 4 shows the immunophenotyping of YAP1 overexpressed mesenchymal stem cells

FIG. 5 shows the multi-lineage differentiation ability of YAP1 overexpressed mesenchymal stem cells

### DETAILED DESCRIPTION

The source of mesenchymal stem cells used in the present invention: the various human tissues used in the present invention were clinical waste or ex vivo human tissues. For example, the placenta in the embodiments was obtained from a maternity ward of The First Affiliated Hospital of Zhejiang University School of Medicine, and all protocols for processing human tissues and cells were approved by the hospital research ethics committee (ethics No.: 2013-272). The technical solutions of the present invention do not involve the specific operations of obtaining human tissues.

If not specifically indicated, the biochemical reagents used in the embodiments are commercially available, and the technical means used in the embodiments are conventional means known to those skilled in the art.

Experimental Apparatus and Reagents

Centrifuge tube (corning, America), centrifuge (eppendorf, Germany), 10 cm culture dish (Greiner, Germany), 100 μm sieve (corning, America), thermostatic shaker (Thermo, America), inverted microscope (Nikon, Japan), RTCA S16 Analyzer (ACEA, America), culture flasks (corning, America), CO₂ incubator (Thermo, America), vertical electrophoresis instrument (Bio-Rad, America), electrotransfer membrane instrument (Bio-Rad, America), chemiluminescence imaging system (Qinxiang, China), BD LSR II flow cytometer (BD, America)

Collagenase IV (invitrogen, America), DMEM (Gibco, America), PBS (JINUO, China), polybrene (GenePharma, China), BCA kit (Thermo, America), RIPA lysate solution (Biyuntian, China), PVDF membrane (Millipore America), YAP1 antibody (Abcam, UK), GAPDH antibody (Abcam, UK), YAP1 lentiviral vector (Jiman Biotechnology, China), YAP1 plasmid vector (Jiman Biotechnology, China).

The technical solutions of the present invention are described in detail below in combination with specific examples.

1. Isolation and Culture of Placenta-Derived Mesenchymal Stem Cells

The isolation and culture are carried out as follows.

(1) A small piece of placenta tissue was cut and washed with phosphate buffer saline (PBS) on a 10 cm culture dish until the placental tissue become light pink (the washing solution should be clear at this time).

(2) The washed tissue was placed on another 10 cm culture dish and sufficiently chopped with a clean surgical scissor.

(3) The chopped tissue pieces were transferred to a 50 ml centrifuge tube, added 25 ml of collagenase IV at a concentration of 0.1% (mass/volume), and digested by shaking on a thermostatic shaker at 37° C. for 30 minutes.

(4) 20 ml of phosphate buffer saline (PBS) was added to the digested tissue, mixed well and filtered through a 100 μm sieve.

(5) The harvested filtrate was centrifuged at 1200 rpm for 5 minutes, and a supernatant was removed and a cell precipitate was retained.

(6) 5 ml of DMEM medium containing 20% fetal bovine serum was added to the cell precipitate, mixed well and inoculated into a T25 flask, placed in a constant temperature incubator with 5% CO₂ at 37° C., and then replaced with a fresh DMEM medium containing 20% fetal bovine serum every 3 days.

2. Cell Transfection

Cells were inoculated on a culture plate and randomly divided into an overexpression control group and an overexpression group.

The overexpression control group was transfected with a lentivirus-null, and the overexpression group was transfected with a lentivirus containing the YAP1 gene (SEQ ID NO: 1). Transfection was performed according to the instructions of lentiviral transfection. After 2-3 days of lentivirus transfection, cells were observed for the expression of green fluorescent protein (GFP) by fluorescence microscopy. When the expression of green fluorescent protein (GFP) was strongest, a complete medium containing puromycin without virus was used for screening, and cells that were not successfully transfected with lentivirus were killed. Successfully transfected cells can be further passaged and cultured.

3. Determination of the Expression of YAP1 in Two Groups of Cells by Western Blot

After transfection treatment, all groups of cells were collected, washed with phosphate buffer solution (PBS) and then lysed with protein lysis solution (RIPA), centrifuged at 12000 rpm for 20 minutes, and a supernatant was collected and the protein content in the supernatan was measured by a BCA kit. The SDS-PAGE electrophoresis was performed with a sample amount of 30 μg of protein. The proteins were transferred to PVDF membrane by a wet transfer printing method, closed with 50g/ml skim milk powder solution at room temperature for 2h, added YAP1 and GAPDH primary antibody dilution, overnight at 4° C.; TBST was used for washing the membrane, added secondary antibody and shaked gently at room temperature for 2h; TBST buffer was used for rinsing fully, added chemiluminescence substrate for reaction, and photographed by chemiluminescence analyzer.

4. Proliferation ability of mesenchymal stem cells after YAP1 overexpression.

The proliferation ability of mesenchymal stem cells was determined by measuring cell viability using a real-time cellular electronic sensing system (RTCA S16 Analyzer), which measures cell status (referred to as “cell index”) based on electrical impedance, and a resistance correlates with cell morphology, adhesion and viability. When a battery is attached to the bottom of a plate coated with electrodes, a change in the local ionic environment occurs, resulting in an increase in impedance.

Measurements are performed according to the supplier's instructions, comprising: a cell culture medium (100 μL) containing 4×10³ cells was loaded into each well of a 16-well plate. The plate was incubated at room temperature for at least 30 minutes and then inserted into the system. Cell proliferation was monitored in real time for 100 hours. The population doubling time of cells may be analyzed by RTCA Data Analysis Software 1.0 software.

5. Surface Markers of Mesenchymal Stem Cells After YAP1 Overexpression

The cells were harvested and washed with phosphate-buffered saline. The concentration of cells was adjusted to 1×10⁶ cells per 100 μl. The cells were then incubated with phycoerythrin—conjugated antibodies in darkness at room temperature for 30 min and then washed with PBS. Expression of surface antigens was analyzed using a BD LSR II flow cytometer.

6. Multi-Lineage Differentiation Ability of Mesenchymal Stem Cells After YAP1 Overexpression

For adipogenic, osteogenic or chondrogenic differentiation, cells were plated at a density of 1×10³/cm² on six-well multidishes. Cells were exposed to adipogenic, osteogenic or chondrogenic induction medium for 21 days. Adipogenic, osteogenic, chondrogenic differentiation can be assessed by Oil Red O staining, Alizarin Red S staining, and alcian blue staining, respectively.

Experimental Results

1. Results of Lentivirus Transfection

The Western Blot results (FIG. 1) showed that the expression of YAP1 in the overexpression group was significantly up-regulated, indicating that a cell model of YAP1 overexpression was successfully established and achieved increased expression of YAP1.

2. Proliferation Ability of Mesenchymal Stem Cells After YAP1 Overexpression

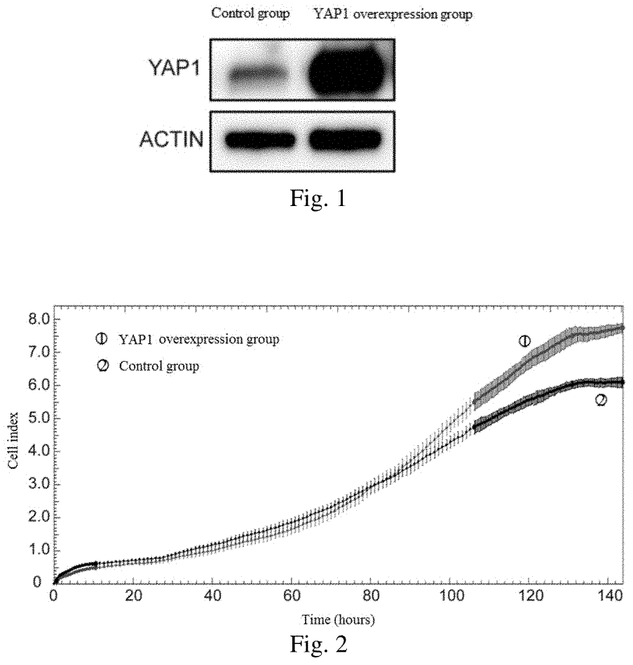

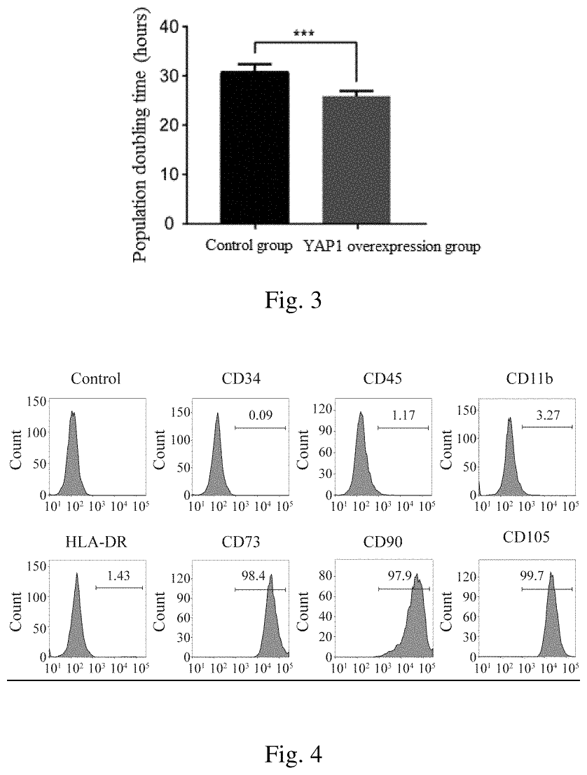

As shown by the value-added curves (FIG. 2) measured in the real-time monitoring system and the statistical analysis results (FIG. 3) that the proliferation rate of placental mesenchymal stem cells was significantly accelerated after YAP1 overexpression, and the population doubling time was significantly reduced.

3. The Other Characteristic of Mesenchmal Stem Cells After YAP1 Overexpression

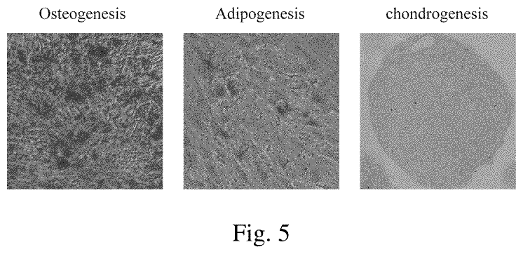

As shown by the the results of flow cytometry (FIG. 4) and the stained pictures after differentiation (FIG. 5) , the YAP1 overexpressed placental mesenchymal stem cells still has the common surface markers and multi-lineage differentiation ability of mesenchmal stem cells.

The gene modification method in the present invention can not only use the YAP1-overexpressing lentiviral vector, but also an YAP1-overexpressing plasmid vector. At the same time, the source of the primary mesenchymal stem cells can also be umbilical cord or adipose tissue. In view of the mastery of corresponding technical means by those skilled in the art, repeated descriptions of specific elements of these optional schemes are omitted in the present invention.

What is claimed is:

1. A method of preparing the YAP1 gene-modified mesenchymal stem cell, comprising:
(1) taking small pieces of placenta, umbilical cord or adipose tissue containing a primary mesenchymal stem cell; washing with phosphate buffer saline on a culture dish until a washing solution becomes clear, and then sufficiently cutting up;
(2) transferring the chopped tissue pieces to a 50 ml centrifuge tube, adding 25 ml of collagenase IV at a mass/volume concentration of 0.1%, and digesting by shaking on a shaker at a constant temperature of 37° C. for 30 minutes;
(3) adding 20 ml of phosphate buffer saline to the digested tissue, mixing well and filtering through a 100 pm sieve to obtain a filtrate; centrifuging the filtrate at 1200 rpm for 5 minutes to obtain a supernatant, and removing the supernatant;
(4) adding 5 ml of DMEM medium containing 20% fetal bovine serum to the cell precipitate, mixing well and seeding into a T25 culture flask; then placing in an incubator with 5% CO₂ and a constant temperature of 37° C.; replacing with a fresh DMEM medium containing 20% fetal bovine serum every 3 days; and
(5) after the cell have reached 50% confluence, adding a YAP1 lentiviral vector or a YAP1 plasmid vector at a multiplicity of infection of 50:1 to transfect the mesenchymal stem cell; after completing transfection, a primary mesenchymal stem cell YAP1-LV-MSC modified by overexpressed YAP1 gene is successfully prepared, wherein the YAP1 lentiviral vector or a YAP1 plasmid vector contains a YAP1 gene coding sequence of SEQ ID NO: 1.
